(19) United States
(12) Patent Application Publication
(10) Pub. No.: US 2009/0264475 A1
(43) Pub. Date: Oct. 22, 2009

(54) METHODS AND COMPOSITIONS FOR ALTERING DIETARY BEHAVIOR

(76) Inventor: Daniel M. Schwartz, San Francisco, CA (US)

Correspondence Address: FULBRIGHT & JAWORSKI, LLP, 1301 MCKINNEY, SUITE 5100, HOUSTON, TX, 77010-3095, US

(21) Appl. No.: 12/358,321

(22) Filed: Jan. 23, 2009

Related U.S. Application Data
(60) Provisional application No. 61/023,205, filed on Jan. 24, 2008.

(51) Int. Cl. A61K 31/465; A23K 1/18

(52) U.S. Cl. 514/343; 426/2

(57) ABSTRACT

The present invention concerns exploiting a characteristically negative behavior, such as habitual behavior or addiction, for example, for a beneficial purpose. In particular aspects, an individual desires eating healthy comestibles that comprises a positively-reinforcing compound, thereby consuming greater quantities of one or more healthy comestibles. In particular aspects, an obese or overweight individual loses weight and/or lowers BMI by consuming at least one healthy comestibles comprising the positively-reinforcing compound. In other aspects, an individual with a medical condition that could benefit from one or more particular comestibles consumes the one or more particular comestibles comprising a positively-reinforcing compound.

This application claims priority to U.S. Provisional Patent Application Ser. No. 61/023,205, filed Jan. 24, 2008, which is incorporated by reference herein in its entirety.

### FIELD OF THE INVENTION

The present invention generally concerns the fields of nutrition and medicine. In particular, the invention concerns fields related to modified foods for the treatment of medical conditions, such as obesity, for example.

### BACKGROUND OF THE INVENTION

Many people with obesity suffer morbidity and premature mortality. In spite of the adverse effects of obesity, it is very difficult for affected patients to alter dietary behavior. Not only do obese people have increased consumption, but they often consume unhealthy foods, such as those comprising high levels of fats and processed sugars. Filling up on these foods, they consume less healthier ones, such as fruits and vegetables. High consumption of foods comprising fat and/or sugar promotes development of heart disease, diabetes, and cancer. There is a strong need to develop means to prompt individuals to eat healthier foods.

### BRIEF SUMMARY OF THE INVENTION

In certain aspects of the invention, an addictive agent that is characteristically considered to be deleterious is exploited for beneficial purposes. The treated individual may be an adult, an adolescent, or a child. In embodiments of the invention, the compositions are used to alter dietary behavior. For example, there are methods and compositions that encourage an individual to eat nutritious foods, for example. In specific embodiments, the individual is an obese individual, although in alternative embodiments the individual is not obese, such as an individual that is overweight, an individual with poor nutritious habits, and/or an individual that desires to lose weight and/or reduce body mass index (BMI). In additional embodiments, the individual is not obese but needs encouragement to eat particular foods, such as vegetables and fruit to promote better health, milk to increase bone strength and/or density, and a variety of foods to prevent and/or treat heart disease and/or cancer. In particular, the methods and compositions of the invention concern encouraging an individual to become addicted to one or more healthy foods. For example, the individual may be encouraged to eat one or more particular foods to treat obesity, treat heart disease, diabetes, degenerative arthritis, improve eyesight, and/or enhance bone growth and/or density.

In specific embodiments, one or more addictive compounds are incorporated by any suitable method into one or more foods that are healthy. In certain aspects, such behavior results in the individual losing weight and/or results in the individual being treated for a medical condition or prevention thereof.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### DETAILED DESCRIPTION OF THE INVENTION

### I. Definitions

In keeping with long-standing patent law convention, the words “a” and “an” when used in the present specification in concert with the word comprising, including the claims, denote “one or more.” Some embodiments of the invention may consist of or consist essentially of one or more elements, method steps, and/or methods of the invention. It is contemplated that any method or composition described herein can be implemented with respect to any other method or composition described herein.

The term “addictive” as used herein refers to compulsive physiological need for a habit-forming substance.

The term “elevated amount of a positively-reinforcing composition” as used herein refers to an amount of a positively-reinforcing composition in a comestible that is greater than the amount that is native to the comestible. In specific embodiments, the amount is sufficient to establish and/or maintain a desire in the individual to consume more of the comestible. In some embodiments, the amount that is native to the comestible may be substantially zero and undetectable by standard means. In this case, the elevated amount of a positively-reinforcing composition is greater than zero and detectable by standard means.

The terms “habitual” or “habituating” as used herein refer to the act or process of making an event the nature of a recurrent pattern of behavior.

The term “healthy comestible”, which may also be referred to as a “healthy food,” as used herein is a food that will increase the health of the individual. It can do so either by being intrinsically healthy or by promoting satiety such that less healthy foods are consumed in reduced quantities.

The term “obese” as used herein refers to an individual that has a body mass index (BMI) of 30 or higher. An adult who has a BMI between 25 and 29.9 is considered overweight.

The term “positively-reinforcing composition” as used herein refers to a composition that affects an individual such that the individual desires more consumption of the composition. In specific embodiments, the positively-reinforcing composition comprises a habit-forming composition, such as an addictive composition, for example.

### II. The Present Invention

In general embodiments, the present invention concerns altering the behavior of an individual to consume one or more particular foods. In some embodiments, the invention concerns a means, such as adding addictive compositions to foods that induce habitual behavior or addiction, to promote individuals to consume healthier foods, and in specific embodiments in preference to those that are high in fat and/or processed sugars. Individuals are driven to eat the healthier foods because they have added agent(s) that is positively reinforcing and, in specific embodiments, is addictive. Because the patient becomes addicted to the additive, he/she consumes significant quantities of the desired food. As appetite is satisfied either partially or completely by the foods comprising the positively-reinforcing agent(s), and there is diminished ingestion of fatty foods and those containing processed sugars that do not contain the addictive composition(s).

The addictive agent can be placed in any food, whether it is “healthy” or not. In any event, it promotes eating the particular food or foods that comprise it.

### III. Exemplary Positively-Reinforcing Compounds

In particular embodiments, one or more positively-reinforcing compounds are incorporated into one or more foods, such as for the encouragement of an individual to consume healthier foods. The positively-reinforcing compound may be of any suitable kind so long as it is suitably desirous to the individual to instill desire for additional doses of the positively-reinforcing compound, for example the addictive agent incorporated into a healthy food.

Nevertheless, exemplary addictive agents comprise nicotine, opioids, ephedrine, caffeine, kavalactones, pshychostimulants, cannabis, 3,4 methylenedioxymethamphetamine (MDMA), catha edulis, Dextromethorphan (DXM), celexa, Mitragynine speciosa (“Krathom”), and Betel quid, for example.

### IV. Exemplary Foods Comprising One or More Positively-Reinforcing Compounds

In specific embodiments, one or more particular foods are modified such that they comprise one or more positively-reinforcing compounds. In cases wherein the positively-reinforcing compound may be naturally present in the food, the amount or production of the endogenous addictive agent may be increased. In particular embodiments, the food is a healthy food. The food may be of any kind so long as modifying it to comprise an addictive agent results in healthful benefits to the consumer. In certain embodiments, if the positively-reinforcing compound is naturally present in the food, then the level of the compound will be greater than is normally found.

In particular aspects of the invention, the food comprises one or more vegetables, one or more fruits, one or more lean meats, one or more whole grains, legumes, or any other food whose consumption is beneficial to the individual.

Vegetables suitable for modification in accordance with the invention include spinach, broccoli, mustard greens, collard greens, green beans, kale, carrots, zucchini, cauliflower, corn, lettuce, tomato, onion, asparagus, cabbage, turnips, radish, brussel sprouts, or rhubarb, for example.

Fruits suitable for modification in accordance with the invention include oranges, strawberries, bananas, apples, peaches, nectarines, mangoes, grapes (white or red), plums, lemons, limes, apricots, prunes, dates, grapefruit, tangerine, passion fruit, pineapple, cherry, blueberry, gooseberry, cranberry, cranapple, dewberry, or blackberry, for example.

Lean meats suitable for modification in accordance with the invention include chicken, turkey, pork, or venison, for example.

Whole grains suitable for modification in accordance with the invention include oat, wheat, corn, rye, rice, millet, barley, or buckwheat, for example.

Dairy products suitable for modification in accordance with the invention include milk, cheese, non-fat or low-fat yogurt, cottage cheese, ice cream, although low-fat or low calorie ice cream, in specific embodiments, for example.

Legumes suitable for modification in accordance with the invention include peas, lentils, beans, or nuts, for example.

By way of example, vegetables such as spinach can be treated with known quantities of nicotine, a well studied addictive substance approved for oral ingestion (Nicorette™ gum). Other healthy vegetables and fruits can be similarly treated. As a nicotine addiction is established from continued ingestion, patients will consume certain minimal amounts of the healthy produce each day to satisfy the nicotine addiction. These foods will supplant other ones containing high fat or processed sugar content that do not contain nicotine. By replacing fatty and sugar containing foods with addictive vegetables and fruits, patients will lose weight, lower serum lipid levels, and be less prone to diabetes, hypertension, and heart disease.

While not limited to this particular method, one means to initially establish the nicotine addiction could use nicotine-comprising gum (Nicorette™) or candy. Then, once established, the addiction would be satisfied with nicotine-comprising foods. Such foods do not necessarily have to, but could be, administered under the supervision of a physician with specific quantities of nicotine consumed each day based on a prescription. Foods comprising an added agent used to manage a disease are called “medical food,” and specific embodiments of the invention encompass a medical food. Medical foods as defined in section 5(b) of the Orphan Drug Act (21 U.S.C. 360ee(b)(3)) (From the Office of the Federal Register, National Archives and Records Administration) are as follows: a medical food is a food that is formulated to be consumed or administered enterally under the supervision of a physician and that is intended for the specific dietary management of a disease or condition for which distinctive nutritional requirements, based on recognized scientific principles, are established by medical evaluation. A food is subject to this exemption only if:

i. It is a specially formulated and processed product (as opposed to a naturally occurring foodstuff used in its natural state) for the partial or exclusive feeding of a patient by means of oral intake or interal feeding by tube;

ii. It is intended for the dietary management of a patient who, because of therapeutic or chronic medical needs, has limited or impaired capacity to ingest, digest, absorb, or metabolize ordinary foodstuffs or certain nutrients, or who has other special medically determined nutrient requirements, the dietary management of which cannot be achieved by the modification of the normal diet alone;

iii. It provides nutritional support specifically modified for the management of the unique nutrient needs that result from the specific disease or condition, as determined by medical evaluation;

iv. It is intended to be used under medical supervision; and

v. It is intended only for a patient receiving active and ongoing medical supervision wherein the patient requires medical care on a recurring basis for, among other things, instructions on the use of the medical food.

The added positively-reinforcing compound need not be restricted to nicotine. Other addictive agents can be used alone or in combination with nicotine as long as they are not associated with untoward events in significant numbers of patients.

### V. Preparation of the Comestible

In certain aspects of the invention, a healthy comestible comprising a positively-reinforcing compound is obtained for consumption by an individual. In some aspects the comestible is obtained through prescription by a health care provider and, optionally, a pharmacist. In other aspects the comestible is obtained by commercial means, such as through a market or grocery store.

The positively-reinforcing compound may be incorporated into the comestible by any suitable method so long as an effective amount of the positively-reinforcing compound is ingested through consumption by the consumer. The positively-reinforcing compound may be in the comestible, on the comestible, and/or added to the comestible by the consumer. The comestible may be submerged in a solution or other composition comprising the positively-reinforcing compound, whereupon an effective amount of the positively-reinforcing compound soaks onto and/or into the comestible that is ultimately consumed.
A. Incorporation of the Comestible by Genetic Transfer

In specific embodiments of the invention, the healthy comestible comprising the positively-reinforcing compound is a genetically modified food. In particular, a healthy comestible that does not comprise a sufficient amount of the positively-reinforcing compound, for example, is manipulated by genetic transfer to comprise a polynucleotide that encodes a gene product that indirectly or directly increases the amount of positively-reinforcing compound in the now-manipulated healthy comestible.

1. Plant Transformation Constructs

The construction of vectors that may be employed in conjunction with plant transformation techniques according to the invention will be known to those of skill of the art (see for example, Sambrook et al., 1989; Gelvin et al., 1990).

In specific aspects of the invention, a transformation construct comprising at least one polynucleotide that is suitable to directly or indirectly produce a positively-reinforcing compound or to increase the level of an endogenous positively-reinforcing compound in the target plant. In some embodiments, the polynucleotide encodes a gene product that itself produces the positively-reinforcing compound, such as an enzyme in a pathway that produces it, for example. In other embodiments, the polynucleotide encodes a gene product that is upstream from an enzyme that produces the positively-reinforcing compound. In certain aspects, both gene products are encoded by the polynucleotide. In additional embodiments, there is a polynucleotide that encodes a gene product that shunts an existing or new pathway into a favorable direction to produce a positively-reinforcing compound.

In certain embodiments, the present inventors contemplate the transformation of a recipient cell with one or more transformation constructs. Two or more transgenes can be created in a single transformation event using either distinct selected-protein encoding vectors, or using a single vector incorporating two or more gene coding sequences, for example. In addition to the transgene associated with the present invention, another type of transgene may be employed, such as one conferring, for example, herbicide, insect, disease (viral, bacterial, fungal, nematode) or drought resistance, male sterility, drydown, standability, prolificacy, starch properties, oil quantity and quality, or those increasing yield or nutritional quality may be employed as desired.

In other embodiments of the invention, it is contemplated that one may wish to employ replication-competent viral vectors for plant transformation. Such vectors include, for example, wheat dwarf virus (WDV) “shuttle” vectors, such as pW1-11 and PW1-GUS (Ugaki et al., 1991). These vectors are capable of autonomous replication in maize cells as well as E. coli, and as such may provide increased sensitivity for detecting DNA delivered to transgenic cells. A replicating vector also may be useful for delivery of genes flanked by DNA sequences from transposable elements such as Ac, Ds, or Mu. It has been proposed that transposition of these elements within the maize genome requires DNA replication (Laufs et al., 1990). It also is contemplated that transposable elements would be useful for introducing DNA fragments lacking elements necessary for selection and maintenance of the plasmid vector in bacteria, e.g., antibiotic resistance genes and origins of DNA replication. It also is proposed that use of a transposable element such as Ac, Ds, or Mu would actively promote integration of the desired DNA and hence increase the frequency of stably transformed cells.

Vectors used for plant transformation may include, for example, plasmids, cosmids, YACs (yeast artificial chromosomes), BACs (bacterial artificial chromosomes) or any other suitable cloning system. It is contemplated that utilization of cloning systems with large insert capacities will allow introduction of large DNA sequences comprising more than one selected gene, if desired. Introduction of such sequences may be facilitated by use of bacterial or yeast artificial chromosomes (BACs or YACs, respectively), or even plant artificial chromosomes. For example, the use of BACs for Agrobacterium-mediated transformation was disclosed by Hamilton et al. (1996).

Particularly useful for transformation are expression cassettes that have been isolated from such vectors. DNA segments used for transforming plant cells will, of course, generally comprise the cDNA, gene or genes which one desires to introduced into and have expressed in the host cells. These DNA segments can further include structures such as promoters, enhancers, polylinkers, and/or even regulatory genes as desired. The DNA segment or gene chosen for cellular introduction will often encode a protein that will be expressed in the resultant recombinant cells. Preferred components likely to be included with vectors used in the current invention are as follows.

a. Regulatory Elements

Transformation constructs comprising a promoter operably linked to a selected coding region are contemplated. By including an enhancer sequence with such constructs, the expression of the selected protein may be enhanced. These enhancers often are found 5′ to the start of transcription in a promoter that functions in eukaryotic cells, but can often be inserted in the forward or reverse orientation 5′ or 3′ to the coding sequence. In some instances, these 5′ enhancing elements are introns. Deemed to be particularly useful as enhancers are the 5′ introns of the rice actin 1 and rice actin 2 genes. Examples of other enhancers which could be used in accordance with the invention include elements from the CaMV 35S promoter, octopine synthase genes (Ellis et al., 1987), the maize alcohol dehydrogenase gene, the maize shrunken 1 gene and promoters from non-plant eukaryotes (e.g., yeast; Ma et al., 1988).

Where an enhancer is used in conjunction with a promoter for the expression of a selected protein, it is believed that it will be preferred to place the enhancer between the promoter and the start codon of the selected coding region. However, one also could use a different arrangement of the enhancer relative to other sequences and still realize the beneficial properties conferred by the enhancer. For example, the enhancer could be placed 5′ of the promoter region, within the promoter region, within the coding sequence (including within any other intron sequences which may be present), or 3′ of the coding region.

In addition to introns with enhancing activity, other types of elements can influence gene expression. For example, untranslated leader sequences have been made to predict optimum or sub-optimum sequences and generate “consensus” and preferred leader sequences (Joshi, 1987). Preferred leader sequences are contemplated to include those which have sequences predicted to direct optimum expression of the attached coding region, i.e., to include a preferred consensus leader sequence which may increase or maintain mRNA stability and prevent inappropriate initiation of translation. The choice of such sequences will be known to those of skill in the art in light of the present disclosure. Sequences that are derived from genes that are highly expressed in plants, and in maize in particular, will be most preferred.

Specifically contemplated for use in accordance with the present invention are vectors that include the ocs enhancer element. This element was first identified as a 16 bp palindromic enhancer from the octopine synthase (ocs) gene of Agrobacterium (Ellis et al., 1987), and is present in at least 10 other promoters (Bouchez et al., 1989). It is proposed that the use of an enhancer element, such as the ocs element and particularly multiple copies of the element, may be used to increase the level of transcription from adjacent promoters when applied in the context of monocot transformation.

In some embodiments, it is envisioned that a particular use of the present invention may be the production of transformants comprising a transgene that is expressed in a tissue-specific manner. In some embodiments, expression of an antisense transcript would prevent accumulation of a particular gene product encoded by a sense transcript.

It also is contemplated that it may be useful to target DNA within a cell. For example, it may be useful to target introduced DNA to the nucleus as this may increase the frequency of transformation. Within the nucleus itself, it would be useful to target a gene in order to achieve site specific integration. For example, it would be useful to have a gene introduced through transformation replace an existing gene in the cell.

b. Terminators

Transformation constructs prepared in accordance with the invention will typically include a 3′ end DNA sequence that acts as a signal to terminate transcription and allow for the poly-adenylation of the mRNA produced by coding sequences operably linked to the maize GRP promoter. One type of terminator that may be used is a terminator from a gene encoding the small subunit of a ribulose-1,5-bisphosphate carboxylase-oxygenase (rbcS), and more specifically, from a rice rbcS gene. Where a 3′ end other than an rbcS terminator is used in accordance with the invention, the most preferred 3′ ends are contemplated to be those from the nopaline synthase gene of Agrobacterium tumefaciens (nos 3′ end) (Bevan et al., 1983), the terminator for the T7 transcript from the octopine synthase gene of Agrobacterium tumefaciens, and the 3′ end of the protease inhibitor I or II genes from potato or tomato. Regulatory elements such as Adh intron (Callis et al., 1987), sucrose synthase intron (Vasil et al., 1989) or TMV omega element (Gallie, et al., 1989), may further be included where desired. Alternatively, one also could use a gamma coixin, oleosin 3 or other terminator from the genus Coix.

C. Transit or Signal Peptides

Sequences that are joined to the coding sequence of an expressed gene, that are removed post-translationally from the initial translation product and that facilitate the transport of the protein into or through intracellular or extracellular membranes, are termed transit (usually into vacuoles, vesicles, plastids and other intracellular organelles) and signal sequences (usually to the endoplasmic reticulum, golgi apparatus and outside of the cellular membrane). By facilitating the transport of the protein into compartments inside and outside the cell, these sequences may increase the accumulation of gene product protecting them from proteolytic degradation. These sequences also allow for additional mRNA sequences from highly expressed genes to be attached to the coding sequence of the genes. Since mRNA being translated by ribosomes is more stable than naked mRNA, the presence of translatable mRNA in front of the gene may increase the overall stability of the mRNA transcript from the gene and thereby increase synthesis of the gene product. Since transit and signal sequences are usually post-translationally removed from the initial translation product, the use of these sequences allows for the addition of extra translated sequences that may not appear on the final polypeptide. It further is contemplated that targeting of certain proteins may be desirable in order to enhance the stability of the protein (U.S. Pat. No. 5,545,818, incorporated herein by reference in its entirety).

Additionally, vectors may be constructed and employed in the intracellular targeting of a specific gene product within the cells of a transgenic plant or in directing a protein to the extracellular environment. This generally will be achieved by joining a DNA sequence encoding a transit or signal peptide sequence to the coding sequence of a particular gene. The resultant transit, or signal, peptide will transport the protein to a particular intracellular, or extracellular destination, respectively, and will then be post-translationally removed.

d. Plant Promoters

Promoters that are useful for plant transgene expression include those that are inducible, viral, synthetic, constitutive as described (Poszkowski et al., 1989; Odell et al., 1985), temporally regulated, spatially regulated, and spatio-temporally regulated (Chau et al., 1989).

A number of plant promoters have been described with various expression characteristics. Examples of some constitutive promoters that have been described include the rice actin 1 (Wang et al., 1992; U.S. Patent No. 5,641,876), CaMV 35S (Odell et al., 1985), CaMV 19S (Lawton et al., 1987), nos (Ebert et al., 1987), Adh (Walker et al., 1987), sucrose synthase (Yang & Russell, 1990).

Examples of tissue specific promoters which have been described include the lectin (Vodkin et al., 1983; Lindstrom et al., 1990), corn alcohol dehydrogenase 1 (Vogel et al., 1989; Dennis et al., 1984), corn light harvesting complex (Simpson, 1986; Bansal et al., 1992), corn heat shock protein (Odell et al., 1985; Rochester et al., 1986), pea small subunit RuBP carboxylase (Poulsen et al., 1986; Cashmore et al., 1983), Ti plasmid mannopine synthase (Langridge et al., 1989), Ti plasmid nopaline synthase (Langridge et al., 1989), petunia chalcone isomerase (Van Tunen et al., 1988), bean glycine rich protein 1 (Keller et al., 1989), truncated CaMV 35s (Odell et al., 1985), potato patatin (Wenzler et al., 1989), root cell (Conkling et al., 1990), maize zein (Reina et al., 1990; Kriz et al., 1987; Wandelt and Feix, 1989; Langridge and Feix, 1983; Reina et al., 1990), globulin-1 (Belanger and Kriz et al., 1991), beta-tubulin, cab (Sullivan et al., 1989), PEPCase (Hudspeth & Grula, 1989), R gene complex-associated promoters (Chandler et al., 1989), and chalcone synthase promoters (Franken et al., 1991).

Inducible promoters which have been described include ABA- and turgor-inducible promoters, the promoter of the auxin-binding protein gene (Schwob et al., 1993), the UDP glucose flavonoid glycosyl-transferase gene promoter (Ralston et al., 1988); the MPI proteinase inhibitor promoter (Cordero et al., 1994), and the glyceraldehyde-3-phosphate dehydrogenase gene promoter (Kohler et al., 1995; Quigley et al., 1989; Martinez et al., 1989).

2. Production and Characterization of Stably Transformed Plants

After effecting delivery of exogenous DNA to recipient cells, the next steps generally concern identifying the transformed cells for further culturing and plant regeneration. In order to improve the ability to identify transformants, one may desire to employ a selectable or screenable marker gene as, or in addition to, the expressible gene of interest. In this case, one would then generally assay the potentially transformed cell population by exposing the cells to a selective agent or agents, or one would screen the cells for the desired marker gene trait.

a. Selection

It is believed that DNA is introduced into only a small percentage of target cells in any one experiment. In order to provide an efficient system for identification of those cells receiving DNA and integrating it into their genomes one may employ a means for selecting those cells that are stably transformed. One exemplary embodiment of such a method is to introduce into the host cell, a marker gene which confers resistance to some normally inhibitory agent, such as an antibiotic or herbicide. Examples of antibiotics which may be used include the aminoglycoside antibiotics neomycin, kanamycin and paromomycin, or the antibiotic hygromycin. Resistance to the aminoglycoside antibiotics is conferred by aminoglycoside phosphostransferase enzymes such as neomycin phosphotransferase II (NPT II) or NPT I, whereas resistance to hygromycin is conferred by hygromycin phosphotransferase.

Potentially transformed cells then are exposed to the selective agent. In the population of surviving cells will be those cells where, generally, the resistance-conferring gene has been integrated and expressed at sufficient levels to permit cell survival. Cells may be tested further to confirm stable integration of the exogenous DNA. Using the techniques disclosed herein, greater than 40% of bombarded embryos may yield transformants.

b. Regeneration and Seed Production

Cells that survive the exposure to the selective agent, or cells that have been scored positive in a screening assay, may be cultured in media that supports regeneration of plants. In an exemplary embodiment, MS and N6 media may be modified (see Table 7) by including further substances such as growth regulators. A preferred growth regulator for such purposes is dicamba or 2,4-D. However, other growth regulators may be employed, including NAA, NAA+2,4-D or perhaps even picloram. Media improvement in these and like ways has been found to facilitate the growth of cells at specific developmental stages. Tissue may be maintained on a basic media with growth regulators until sufficient tissue is available to begin plant regeneration efforts, or following repeated rounds of manual selection, until the morphology of the tissue is suitable for regeneration, at least 2 wk, then transferred to media conducive to maturation of embryoids. Cultures are transferred every 2 wk on this medium. Shoot development will signal the time to transfer to medium lacking growth regulators.

The transformed cells, identified by selection or screening and cultured in an appropriate medium that supports regeneration, will then be allowed to mature into plants. Developing plantlets are transferred to soiless plant growth mix, and hardened, e.g., in an environmentally controlled chamber at about 85% relative humidity, 600 ppm CO2, and 25-250microeinsteins m 2 s-1 of light. Plants are preferably matured either in a growth chamber or greenhouse. Plants are regenerated from about 6 wk to 10 months after a transformant is identified, depending on the initial tissue. During regeneration, cells are grown on solid media in tissue culture vessels. Illustrative embodiments of such vessels are petri dishes and Plant Cons. Regenerating plants are preferably grown at about 19° C. to about 28° C. After the regenerating plants have reached the stage of shoot and root development, they may be transferred to a greenhouse for further growth and testing.

Note, however, that seeds on transformed plants may occasionally require embryo rescue due to cessation of seed development and premature senescence of plants. To rescue developing embryos, they are excised from surface-disinfected seeds 10-20 days post-pollination and cultured. An embodiment of media used for culture at this stage comprises MS salts, 2% sucrose, and 5.5 g/l agarose. In embryo rescue, large embryos (defined as greater than 3 mm in length) are germinated directly on an appropriate media. Embryos smaller than that may be cultured for 1 wk on media containing the above ingredients along with 10-5M abscisic acid and then transferred to growth regulator-free medium for germination.

Progeny may be recovered from transformed plants and tested for expression of the exogenous expressible gene by localized application of an appropriate substrate to plant parts such as leaves. In the case of bar transformed plants, it was found that transformed parental plants (RO) and their progeny of any generation tested exhibited no bialaphos-related necrosis after localized application of the herbicide Basta to leaves, if there was functional PAT activity in the plants as assessed by an in vitro enzymatic assay. All PAT positive progeny tested contained bar, confirming that the presence of the enzyme and the resistance to bialaphos were associated with the transmission through the germline of the marker gene.

C. Characterization

To confirm the presence of the exogenous DNA or “transgene(s)” in the regenerating plants, a variety of assays may be performed. Such assays include, for example, “molecular biological” assays, such as Southern and Northern blotting and PCR™; “biochemical” assays, such as detecting the presence of a protein product, e.g., by immunological means (ELISAs and Western blots) or by enzymatic function; plant part assays, such as leaf or root assays; and also, by analyzing the phenotype of the whole regenerated plant.

d. DNA Integration, RNA Expression and Inheritance

Genomic DNA may be isolated from callus cell lines or any plant parts to determine the presence of the exogenous gene through the use of techniques well known to those skilled in the art. Note, that intact sequences will not always be present, presumably due to rearrangement or deletion of sequences in the cell.

The presence of DNA elements introduced through the methods of this invention may be determined by polymerase chain reaction (PCR™). Using this technique discreet fragments of DNA are amplified and detected by gel electrophoresis. This type of analysis permits one to determine whether a gene is present in a stable transformant, but does not prove integration of the introduced gene into the host cell genome. It is the experience of the inventor, however, that DNA has been integrated into the genome of all transformants that demonstrate the presence of the gene through PCR™ analysis. In addition, it is not possible using PCR™ techniques to determine whether transformants have exogenous genes introduced into different sites in the genome, i.e., whether transformants are of independent origin. It is contemplated that using PCR™ techniques it would be possible to clone fragments of the host genomic DNA adjacent to an introduced gene.

Positive proof of DNA integration into the host genome and the independent identities of transformants may be determined using the technique of Southern hybridization. Using this technique specific DNA sequences that were introduced into the host genome and flanking host DNA sequences can be identified. Hence the Southern hybridization pattern of a given transformant serves as an identifying characteristic of that transformant. In addition it is possible through Southern hybridization to demonstrate the presence of introduced genes in high molecular weight DNA, i.e., confirm that the introduced gene has been integrated into the host cell genome. The technique of Southern hybridization provides information that is obtained using PCR™, e.g., the presence of a gene, but also demonstrates integration into the genome and characterizes each individual transformant.

It is contemplated that using the techniques of dot or slot blot hybridization which are modifications of Southern hybridization techniques one could obtain the same information that is derived from PCR™, e.g., the presence of a gene.

Both PCR™ and Southern hybridization techniques can be used to demonstrate transmission of a transgene to progeny. In most instances the characteristic Southern hybridization pattern for a given transformant will segregate in progeny as one or more Mendelian genes (Spencer et al., 1992) indicating stable inheritance of the transgene.

Whereas DNA analysis techniques may be conducted using DNA isolated from any part of a plant, RNA will only be expressed in particular cells or tissue types and hence it will be necessary to prepare RNA for analysis from these tissues. PCR™ techniques also may be used for detection and quantitation of RNA produced from introduced genes. In this application of PCR™ it is first necessary to reverse transcribe RNA into DNA, using enzymes such as reverse transcriptase, and then through the use of conventional PCR™ techniques amplify the DNA. In most instances PCR™ techniques, while useful, will not demonstrate integrity of the RNA product. Further information about the nature of the RNA product may be obtained by Northern blotting. This technique will demonstrate the presence of an RNA species and give information about the integrity of that RNA. The presence or absence of an RNA species also can be determined using dot or slot blot Northern hybridizations. These techniques are modifications of Northern blotting and will only demonstrate the presence or absence of an RNA species.

e. Gene Expression

While Southern blotting and PCR™ may be used to detect the gene(s) in question, they do not provide information as to whether the gene is being expressed. Expression may be evaluated by specifically identifying the protein products of the introduced genes or evaluating the phenotypic changes brought about by their expression.

Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Assay procedures also may be used to identify the expression of proteins by their functionality, especially the ability of enzymes to catalyze specific chemical reactions involving specific substrates and products. These reactions may be followed by providing and quantifying the loss of substrates or the generation of products of the reactions by physical or chemical procedures. Examples are as varied as the enzyme to be analyzed and may include assays for PAT enzymatic activity by following production of radiolabeled acetylated phosphinothricin from phosphinothricin and 14C-acetyl CoA or for anthranilate synthase activity by following loss of fluorescence of anthranilate, to name two.

Very frequently the expression of a gene product is determined by evaluating the phenotypic results of its expression. These assays also may take many forms including but not limited to analyzing changes in the chemical composition, morphology, or physiological properties of the plant. Chemical composition may be altered by expression of genes encoding enzymes or storage proteins which change amino acid composition and may be detected by amino acid analysis, or by enzymes which change starch quantity which may be analyzed by near infrared reflectance spectrometry. Morphological changes may include greater stature or thicker stalks. Most often changes in response of plants or plant parts to imposed treatments are evaluated under carefully controlled conditions termed bioassays.
B. Incorporation of the Comestible by Additives

In other embodiments of the invention, the healthy comestible comprising the positively-reinforcing compound is produced by incorporating the healthy comestible comprising the positively-reinforcing compound as an additive. The positively-reinforcing compound may be a direct additive that is added to the comestible and/or an indirect additive that becomes part of the food in trace amounts due to its packaging, storage or other handling.

The positively-reinforcing compound may be incorporated during the processing or production of the comestible.

Additives other than the positively-reinforcing compound may be employed in the invention, such as to maintain product consistency; to improve or maintain nutritional value; to maintain palatability and wholesomeness; to provide leavening or control acidity/alkalinity; and/or to enhance flavor or impart desired color.

### VI. Dissemination of the Comestible

The healthy comestible comprising the positively-reinforcing compound may be disseminated to the consumer by any suitable method so long as the individual in need thereof has access to it. For example, the comestible may be obtained commercially, such as at a market or store, it may be ordered over the world wide web and shipped to the individual, and/or it may be obtained from a health care provider, such as, via prescription from the health care provider and pharmacist.

The comestible may obtained in a raw form, such as a raw fruit, meat, vegetable or grain, for example, or it may be obtained in a container, such as a jar, bag, can, package, box, tray, and so forth. If the comestible is in a container, it may be comprised of plastic, paper, foil, cellophane, or a combination thereof, for example. In certain embodiments, the comestible is sterilized and/or comprises additives or preservatives to protect it from spoilage.

The comestible may also be obtained within or among other foods, such as in a mixture of other comestibles, including a prepackaged meal, for example. The other food or foods that accompany the comestible may or may not themselves also be a healthy comestible comprising a positively-reinforcing compound, and the positively-reinforcing compound in the additional comestible may be the same or different than the other comestible. The container may be air-tight.

The comestible may need to be refrigerated after exposure to air from an air-tight container. The comestible may be irradiated prior to packaging and/or following packaging to protect from spoilage, although any modification to the container comprising the comestible must not deleteriously affect the positively-reinforcing compound.

### VII. Measurement of Obesity

In certain embodiments, the individual that consumes the healthy comestible comprising the positively-reinforcing compound is obese. Before, during and/or after the time frame in which the comestible is being consumed by the obese individual, the obesity and/or weight and/or Body Mass Index (BMI) can be measured. An overweight individual or an individual that otherwise desires to lose weight and/or reduce BMI may also be measured in this manner. Weight can be measured by weighing on a scale. BMI for an adult can be calculated using pounds and inches with the following equation: BMI=(Weight in Pounds/(Height in inches)×(Height in inches))×703.

In specific aspects of the invention, an obese individual begins consuming the healthy comestible comprising the positively-reinforcing compound, and the positive reinforcement nature of the compound encourages the obese individual to desire to consume more of the healthy comestible. In specific embodiments, the obese individual begins to lose weight and or develops a healthier BMI directly or indirectly due to the consumption of the comestible. This may be because the hunger and/or eating motivation of the individual becomes satiated by the comestible of the invention.

VIII. Combination Therapy with Exercise

In some embodiments of the invention, an individual that desires to lose weight and/or reduce BMI partakes in exercise in addition to consuming the healthy comestible comprising the positively-reinforcing compound. The exercise may be of any kind and of any frequency. In specific embodiments, the exercise comprises running, jogging, walking, swimming, cycling, tennis, racquetball, weightlifting, basketball, baseball, stairclimbing, yoga, kickboxing, pilates, skiing, or a combination thereof.

### IX. Embodiments of Special Diets

In some embodiments of the present invention, an individual consumes a healthy comestible comprising a positively-reinforcing compound for a reason other than to lose weight or reduce BMI. That is, the methods and compositions of the present invention may be employed to treat and/or prevent one or more other diseases, including cancer, heart disease, eye disorders, bone and/or joint disorders, and so forth. In particular aspects, the individual that consumes the comestibles of the invention is an individual that has been diagnosed with or that is susceptible to or that is suspected of having one or more particular medical conditions.

For example, the individual may be encouraged to eat one or more particular foods to treat and/or prevent heart disease, treat and/or prevent cancer, treat and/or prevent diabetes, improve eyesight, enhance bone growth and/or density, etc. An individual known to be susceptible to cancer or heart disease, such as an individual with personal or family history, may consume the comestible of the invention to treat the condition. In specific embodiments, an individual with a family history of an eye disorder, such as macular degeneration, consumes carrots comprising the positively reinforcing compound. In other embodiments, an individual that has osteoporosis or is susceptible to osteoporosis due to age and gender, for example, consumes one or more dairy products comprising the positively reinforcing compound.

### X. Addressing Addictive Behavior

In certain embodiments, the individual that consumes the healthy comestible comprising the positively-reinforcing compound and/or a health care provider therefore may desire to reduce or eliminate the consumption of the comestible by the individual. In specific embodiments, the individual may need assistance removing the desire for the comestible, such as overcoming the addiction if the compound were an addictive material, for example. In this case, the individual may receive treatment by a health care provider for such, although in alternative embodiments, the individual may obtain assistance without utilizing a health care provider. In specific embodiments, remediation for the addiction may be provided with the comestible itself, although in other embodiments it may be provided separate from the comestible.

In certain examples, one or more compositions are employed to reduce or remove the addition. In specific embodiments, a comestible or another product comprising a reduced amount of the positively-reinforcing compound are utilized for the addiction, and in certain embodiments the comestible comprising the reduced amount of the compound is the same type of comestible as the one that comprised the full and addictive amount. Alternatively, the composition comprising the reduced amount of the positively-reinforcing compound is not the same type of comestible as the one that comprised the full and addictive amount. In specific embodiments, the reduced amount composition comprises a food, gum, or candy, for example. There may be a range of compositions each comprising a different reduced amount of the compound.

In other embodiments, the composition comprising a reduced amount of the positively-reinforcing compound is not to be consumed. For example, the composition may comprise a substrate for transdermal transmission of the reduced amount of the compound, such as a patch, for example. In other embodiments, a reduced amount of the compound may be provided by a pill, syringe, suspension, and so forth.

In other embodiments of the present invention, an addiction is treated by methods other than providing a reduced amount of the addictive compound to the individual. In specific embodiments, the individual is provided a therapy for the addiction, such as a therapy comprising a drug. The drug may be of any suitable kind so long as the addiction is at least improved, although in some embodiments the addiction is cured. Specific examples of drugs include methadone, LAAM, naltrexone, naloxone, and ibogaine.

Additional methods of treating addiction include counseling, psychotherapy, and hypnosis.

### XI.

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Nicotine Addiction for Obesity

A 54 y.o. male with morbid obesity, elevated cholesterol, hypertension and/or type 2 diabetes is unable to lower his weight with dietary and exercise programs. He is unable to avoid binge eating of foods, such as pizza, hamburgers, ice cream, and chocolate chip cookies.

After a thorough physical examination and laboratory studies, the patient is prescribed Nicorette™ gum at a dose of 1 piece comprising about 2-4 mg nicotine, every 1-8 hours. After 2-12 weeks, when an addiction to nicotine is established, the gum ingestion is supplanted by ingestion of fruit and vegetables comprising about 12-60 mg nicotine each day. To consume the required amount of fruits and vegetables in order to “satisfy” the nicotine addiction, treated patients fill up on these healthy foods in lieu of unhealthy ones comprising fats and processed sugars.

In an alternative embodiment, the addictive agent can have the addiction established by an alternative means, such as by a candy, a dissolvable substrate (such as a dissolvable strip), or in some cases a transdermal patch comprising the addictive agent.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of altering dietary behavior in an individual, comprising the step of providing to the individual a healthy comestible comprising an elevated amount of a positively-reinforcing composition.

2. The method of claim 1, wherein the positively-reinforcing composition is further defined as an addictive composition.

3. The method of claim 1, wherein the individual is an obese individual.

4. The method of claim 1, wherein the comestible is a vegetable, a fruit, a whole grain, a lean meat, a dairy product, a legume, a snack food, or a mixture thereof.

5. The method of claim 1, wherein the comestible comprises one or more compounds that are indirectly or directly therapeutic for a medical condition in the individual.

6. The method of claim 5, wherein the medical condition is a bone disorder and the comestible is a dairy product.

7. The method of claim 4, wherein the dairy product is milk or cheese.

8. The method of claim 4, wherein the snack food comprises an energy bar or a low-carb food.

9. The method of claim 1, wherein the providing comprises receiving a prescribed comestible from a physician.

10. The method of claim 1, wherein the positively-reinforcing composition comprises nicotine, capcasaicin, ephedrine, caffeine, codeine, an opioid, a kavalactone, a pshychostimulant, cannabis, 3,4 methylenedioxymethamphetamine (MDMA), catha edulis, Dextromethorphan (DXM), celexa, Mitragynine speciosa (“Krathom”), Betel quid, or a mixture thereof.

11. The method of claim 1, wherein the individual further participates in an exercise regimen.

12. The method of claim 1, wherein the positively-reinforcing composition is comprised within the comestible, on the comestible, or both.

13. The method of claim 4, wherein the vegetable is spinach, broccoli, mustard greens, collard greens, green beans, kale, carrots, zucchini, cauliflower, corn, lettuce, tomato, onion, asparagus, cabbage, turnips, radish, brussel sprouts, or rhubarb.

14. The method of claim 4, wherein the fruit is oranges, strawberries, bananas, apples, peaches, nectarines, mangoes, grapes (white or red), plums, lemons, limes, apricots, prunes, dates, grapefruit, tangerine, passion fruit, pineapple, cherry, blueberry, gooseberry, cranberry, cranapple, dewberry, or blackberry.

15. The method of claim 4, wherein the whole grain comprises oat, wheat, corn, rye, rice, millet, barley, or buckwheat.

16. The method of claim 1, wherein the providing step occurs more than once.

17. The method of claim 1, further comprising the step of providing to the individual a counteractive agent to the positively-reducing composition.

18. The method of claim 17, wherein the counteractive agent comprises a reduced level of the positively-reinforcing composition compared to the level of the positively-reinforcing composition in the comestible.

19. An isolated healthy comestible comprising an elevated amount of a positively-reinforcing composition.

20. The comestible of claim 19, wherein the positively-reinforcing composition is provided to the comestible by the hand of man.

21. The comestible of claim 19, wherein the comestible comprises a vegetable, fruit, whole grain, dairy product, legume, lean meat, or mixture thereof.

22. The comestible of claim 20, wherein the positively-reinforcing composition comprises nicotine, opioids, ephedrine, caffeine, kavalactones, pshychostimulants, cannabis, 3,4 methylenedioxymethamphetamine (MDMA), catha edulis, Dextromethorphan (DXM), celexa, Mitragynine speciosa (“Krathom”), or Betel quid.

23. The comestible of claim 19, wherein said comestible is comprised in a container.

24. The comestible of claim 23, wherein the container is a box, can, jar, bag, tray, or combination thereof.

25. The comestible of claim 19, further comprising a counteractive agent to the positively-reducing composition.

26. The comestible of claim 25, wherein the counteractive agent comprises a reduced level of the positively-reinforcing composition compared to the level of the positively-reinforcing composition in the comestible.

27. A method of producing a healthy comestible comprising an elevated amount of a positively-reinforcing composition, comprising:
providing a healthy comestible; and
introducing one or more positively-reinforcing compositions to the comestible.

28. The method of claim 27, wherein the introducing step comprises subjecting the comestible to the positively-reinforcing composition in a mixture.

29. The method of claim 28, wherein the subjecting is further defined as said composition being on the comestible in the mixture, said composition being in the comestible in the mixture, and/or the comestible being submerged in a solution comprising the composition.

30. The method of claim 27, wherein the introducing step comprises transferring nucleic acid for incorporation into at least one cell of the comestible, upon which the expression of the nucleic acid in the cell indirectly or directly produces an elevated amount of the positively -reinforcing composition in the comestible.

31. The method of claim 30, wherein the incorporation of the nucleic acid into the cell comprises incorporating the nucleic acid into the genome of the cell.
